(19) United States
(12) Patent Application Publication
(10) Pub. No.: US 2026/0108703 A1
(43) Pub. Date: Apr. 23, 2026

(54) NON-RADIALLY EXPANDING CATHETER HAVING AXIALLY SPIRALED RIDGES PRODUCING A NON-UNIFORM OUTER DIAMETER IN AN AXIAL DIRECTION

(71) Applicant: DePuy Synthes Products, Inc., Raynham, MA (US)

(72) Inventors: Chadwin HANNA, JR., Raynham, MA (US); Pedro D. PEDROSO, Parkland, FL (US)

(73) Assignee: DePuy Synthes Products, Inc., Raynham, MA (US)

(21) Appl. No.: 18/921,221

(22) Filed: Oct. 21, 2024

(51) Int. Cl. A61M 25/00

(52) U.S. Cl. CPC A61M 25/005; A61M 25/0012

(57) ABSTRACT

A catheter including a body having a proximal end, an opposite distal end and an outer wall extending axially therebetween, the outer wall defining axially spiraled ridges forming an undulating outer profile non-uniform in outer diameter in an axial direction providing greater trackability by minimizing contact surface friction forces thereby minimizing track force. In certain implementations the catheter may have a non-uniform stiffness profile (i.e., variable flexibility) in the longitudinal/axial direction by varying: (i) differential between maximum outer diameter and minimum outer diameter of the body of the catheter; and/or (ii) ridge pitch representing a separation distance in the longitudinal/axial direction between adjacent axial spiral ridges.

### FIELD

The present disclosure generally relates to catheters used during endovascular medical treatment. In particular, the present disclosure is directed to a catheter, a method for using a catheter and a method for manufacture of a catheter having greater trackability during navigation in the vasculature anatomy by minimizing contact surface friction forces between the exterior surface of the outer wall of the body of the catheter and the inner wall of the vessel or inner wall of a lumen of another catheter (e.g., guide catheter or delivery catheter).

### BACKGROUND

Catheters are widely used today in various endovascular medical treatments. One category of conventional catheters has a radially expandable section transitionable between a radially constricted state and a radially expanded (i.e., enlarged state). For example, funnel catheters have a radially expandable distal section including the distal end/tip particularly well suited in capturing and removing occlusions, blockages or clots. When unsheathed from the guide catheter or delivery catheter, the radially expandable distal section automatically transitions from a radially constricted state to a radially enlarged state. Balloon catheters have a balloon that when inflated with inflation medium radially expands occluding the vessel. Another category of conventional catheters is non-radially expandable remaining in an unchanging single radial state and have a uniform outer diameter in the longitudinal/axial direction. During delivery, the uniform outer diameter in the longitudinal direction of these conventional non-radially expandable catheters maximize direct physical contact along its entire longitudinal/axial length from a proximal end/tip to an opposite distal end/tip with the inner wall of the vessel or inner wall of the lumen of an outer catheter (e.g., a guide catheter or delivery catheter) generating substantial friction force. High track force is required to overcome such significant friction force while advancing the catheter through the vessel of guide catheter thereby hampering trackability.

It would be desirable to develop an improved non-radially expandable catheter with greater trackability by minimizing contact surface friction forces thereby minimizing track force.

### SUMMARY

An aspect of the present disclosure is directed to an improved non-radially expandable catheter with greater trackability by minimizing contact surface friction forces thereby minimizing track force.

Another aspect of the present disclosure relates to an improved non-radially expandable catheter with enhanced trackability by configuring the exterior surface of the longitudinal/axial outer wall of the body of the catheter spanning between the respective proximal and distal ends to have axially spiral ridges producing an outer profile with a non-uniform outer profile in the longitudinal/axial direction.

While yet another aspect of the present disclosure is directed to an improved non-radially expandable catheter having a non-uniform stiffness profile (i.e., variable flexibility) in the longitudinal/axial direction by varying: (i) differential between maximum outer diameter and minimum outer diameter of the body of the catheter; and/or (ii) ridge pitch representing a separation distance in the longitudinal/axial direction between adjacent axial spiral ridges.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is a partial cut away side view prior to collapse and withdraw of the core mandrel during manufacture of the catheter having axial spiral ridges producing an outer profile having a non-uniform outer diameter in an axial direction in accordance with the present disclosure; each of the nested layers (e.g., a liner, a reinforcing filament layer and an outer jacket) of the body of the catheter is depicted in a partial cut away view revealing the layer beneath;

FIG. 2A is a longitudinal cross-sectional view prior to radial collapse and withdraw of the core mandrel during manufacture of the body of the catheter having axial spiral ridges producing an outer profile with a non-uniform outer diameter in a longitudinal/axial direction in accordance with the present disclosure; illustrating each of the nested layers (e.g., a core mandrel, a liner, a reinforcing filament layer and an outer jacket) having the associated axial spiral ridges resulting in an outer profile having a non-uniform outer diameter in the longitudinal/axial direction;

FIG. 2B is a radial cross-sectional view along lines 2B-2B of FIG. 2A;

FIG. 3A is a longitudinal cross-sectional view, following radial collapse and withdraw of the core mandrel, of the completed or finished manufactured composite catheter having axial spiral ridges producing an outer profile with a non-uniform outer diameter in a longitudinal/axial direction in accordance with the present disclosure; depicting each of the assembled nested layers (e.g., a liner, a reinforcing filament layer and an outer jacket) having the associated axial spiral ridges resulting in the outer profile having a non-uniform outer diameter in the longitudinal/axial direction;

FIG. 3B is a radial cross-sectional view along lines 3B-3B of FIG. 3A;

FIG. 4 is a flow chart of the method of manufacturing the body of the catheter having axial spiral ridges producing an outer profile with a non-uniform outer diameter in an axial direction in accordance with the present disclosure;

FIG. 5A is a side view of only the core mandrel in accordance with the present disclosure while in a radially non-collapsed state (i.e., prior to being pulled axially in opposite directions via pull wires) during assembly of the catheter body, depicting the maximum outer diameter of the core mandrel; and

FIG. 5B is a side view of the core mandrel in a radially collapsed state (i.e., pulled axially in opposite directions via the pull wires), depicting the reduced outer diameter of the core mandrel allowing removal from the assembled catheter body.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the term “microcatheter” is a catheter having a diameter that is small in comparison to catheters in cardiovascular applications, i.e. 8 French or less.

As used herein, the term “needle” describes a structure having a sharp pointed end designed to puncture tissue.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

In all the examples illustrated and described herein, the body 100 of the catheter is non-radially expandable and has a plurality of axial spiral ridges 110 producing an outer profile (i.e., an exterior surface of the outer wall spanning between the respective proximal and distal ends) having a non-uniform outer diameter in the longitudinal/axial direction. The non-uniform outer diameter in the longitudinal/axial direction ensures that the contact surface area of the body 100 of the catheter with the inner wall of the vessel or the inner wall of another catheter (e.g., guide catheter or delivery catheter) is exclusively or only at the axial spiral ridges 110. Along the exterior surface of the outer wall 100c in the longitudinal/axial direction, regions interposed between adjacent axial spiral ridges 110 of the body 100 of the catheter do not come into physical contact with (i.e., separated by a predetermined distance from) the inner wall of the vessel or another catheter (e.g., guide catheter or delivery catheter). Restricting, limiting or minimizing the contact surface area along the exterior surface of the body 100 of the catheter exclusively at the axial spiral ridges 110 in turn minimizes the friction force created while navigating through the vessel or through the lumen of another catheter (e.g., guide catheter or delivery catheter). Substantially less friction force is generated with the catheter body having axial spiral ridges in accordance with the present disclosure in comparison to conventional catheters having a uniform outer diameter wherein the contact surface spans the entire length from the proximal to the distal end. Lower generated contact surface friction force reduces track force and ease of trackability while navigating through vasculature.

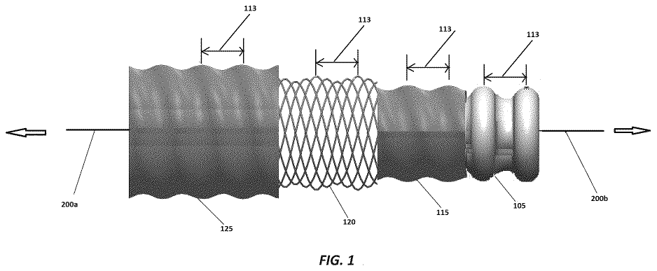

FIG. 1 is a partial cut away side view prior to collapse and withdraw of a core mandrel 105 during manufacture of the body 100 of the catheter forming axial spiral ridges producing an outer profile having a non-uniform outer diameter in the longitudinal/axial direction in accordance with the present disclosure. Manufacture of the body 100 of the catheter starts with providing a pre-formed or otherwise creating a core mandrel 105 forming a plurality of axial spiral ridges 110 producing an outer profile (i.e., outer wall spanning axially between the proximal and distal ends/tips) having a non-uniform outer diameter in the longitudinal/axial direction. In the example in FIG. 1, the axial spiral ridges 110 produce a non-uniform outer diameter extending the full longitudinal/axial length of the body 100 (outer wall 100c) from the proximal end/tip 100a to the opposite distal end/tip 100b. It is, however, possible for the axial spiral ridges 110 to produce a single discrete section of non-uniform outer diameter spanning less than the full longitudinal/axial length of the body 100 of the catheter (i.e., not the full length of outer wall 100c). For instance, the axially spiraled ridges 110 may be present at only a distal section including the distal end/tip 100b of the body 100 of the catheter, while proximal thereto the outer diameter of the body 100 of the catheter may be less than that at the axially spiraled ridges 110 and substantially uniform in the longitudinal/axial direction. In another example, the axially spiraled ridges 110 may be present only along a plurality of discrete sections in the longitudinal/axial direction each having a non-uniform outer diameter in the longitudinal/axial direction, while regions interposed between adjacent discrete sections having the axially spiraled ridges 110 have an outer diameter less than at the axially spiraled ridges 110 and are uniform in the longitudinal/axial direction. The non-uniform outer diameter formed by the axially spiraled ridges fully spanning the longitudinal/axial length of the body 100 of the catheter in the example of FIG. 1 represents a repeating and undulating outer profile in the longitudinal/axial direction, but the non-uniform outer diameter formed by the axially spiraled ridges 110 need neither be repeating nor undulating.

The body 100 of the catheter has a maximum outer diameter at the axially spiraled ridges 110 and a minimum outer diameter at a location in the longitudinal/axial direction between adjacent axially spiraled ridges 110. For example, the minimum outer diameter may, but need not necessarily be, midway (i.e., halfway or midpoint) between adjacent axially spiraled ridges 110. Adjacent axially spiraled ridges 110 are separated by a distance in the longitudinal/axial direction herein referred to as the ridge pitch 130. By changing the ridge pitch 130, the flexibility (i.e., stiffness) of the body 100 of the catheter may be varied in the longitudinal/axial direction, as described in further detail below.

Core mandrel 105 is made of a filament (i.e., metal wire or polymer fiber) made of a biocompatible material. For example, the filament may be made of stainless steel, Polymer ether-ketone (PEEK), Polyethylene terephthalate (PET), nickel titanium (Nitinol), carbon fiber, etc.). Properties associated with each filament may be selected, as desired.

Applied about an exterior surface of the core mandrel 105 is a liner 115 taking on, adopting, identically matching, imparting or conforming to without altering the axially spiraled ridges 110 and the outer profile non-uniform outer diameter in the longitudinal/axial direction of the core mandrel 105. Liner 115 may be coated, wrapped, or applied in any manner about, around or over the exterior surface of the outer wall of the core mandrel 105. Preferably, the liner 115 is a polytetrafluoroethylene (PTFE) coating, but other non-stick material may be used.

Around or about the liner 115 is created a reinforcing filament layer 120 taking on, adopting, identically matching, imparting or conforming to without altering the axially spiraled ridges 110 and the outer profile non-uniform in outer diameter in the longitudinal/axial direction of the liner 115. Reinforcing filament layer 120 includes one or more filaments (e.g., metal wire(s) or polymer fiber(s)) made of a biocompatible material (e.g., stainless steel, Polymer ether-ketone (PEEK), Polyethylene terephthalate (PET), nickel titanium (Nitinol), carbon fiber, etc.) that are braided (i.e., woven) or coiled. The properties for each filament may be selected, as desired. For instance, when using a braided filament, the picks per inch (PPI), number of filaments, diameter of each filament, radial cross-sectional shape (e.g., circular, square, etc.) of each filament may be selected, as desired. Similarly, when using a filament coil, the number of turns and diameter of the filament may be selected, as desired.

Placed (e.g., dip coated, sprayed, applied or wrapped) over the exterior surface of the outer wall (longitudinal/axial wall extending between respective opposite ends) of the reinforcing filament layer 120 is an outer jacket 125. Preferably, outer jacket 125 is a biocompatible polymer material (e.g., heat shrink material) that when subjected to heat secures (e.g., fuses) together the reinforcing filament layer 120 and the liner 115 preventing unraveling of the reinforcing filament layer 120 that otherwise poses a risk of damage to the tissue. Outer jacket 125 takes on the shape of (i.e., adopts, identically matches, conforms, imparts or configured to) the axially spiraled ridges 110 forming the outer profile non-uniform in outer diameter in the longitudinal/axial direction as defined in the outer wall the reinforcing filament layer 120.

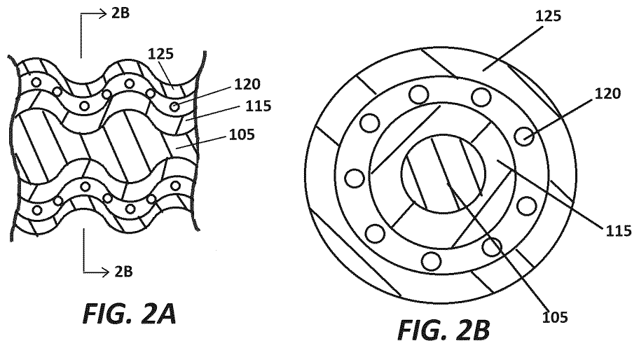

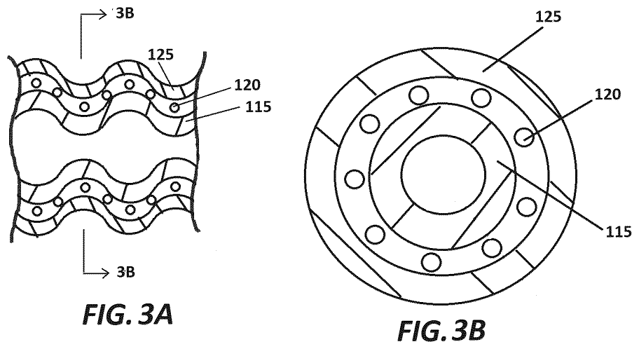

Now that all the layers have been applied, the core mandrel 105 may be withdrawn to form the completed, finished manufacture composite catheter. Prior to withdraw, the core mandrel 105 is radially collapsed (reduced in outer diameter). For example, the interventionalist or physician may initiate radial collapsed by pulling in opposing directions on pull wires 200a, 200b one secured to each of the respective proximal and distal ends of the core mandrel 105. Radial collapse of the core mandrel 105 is fostered or aided via easy release from the liner 115 made of a non-stick material (e.g., PTFE). While in a radially constricted (i.e., radially collapsed) state, the core mandrel 105 may be easily withdrawn (i.e., removed) leaving behind what is hereinafter referred to as the manufactured composite body 100 of the catheter (i.e., including the liner 115 and the reinforcing filament layer 120 secured together via the outer jacket 125). In accordance with the present disclosure, the finished or completed manufactured composite body 100 of the catheter maintains the axial spiral ridges 110 producing the outer profile having a non-uniform outer diameter in the longitudinal axial direction of the exterior surface. Moreover, each individual layer of the manufactured composite body 100 of the catheter maintains along both an interior surface and an exterior surface identically (matching) configured axially spiraled ridges 110 producing a non-uniform outer diameter in the longitudinal/axial direction along both the inner profile (i.e., interior surface) and outer profile (i.e., exterior surface) (FIGS. 2A, 2B, 3A & 3B). In the particular example of FIG. 1, the non-uniform in the longitudinal/axial direction outer profile of the manufactured composite body 100 of the catheter is undulating and repeating, but need not necessarily be either undulating and/or repeating.

With conventional non-radially expanding catheters having a uniform outer diameter extending from a proximal end to an opposite distal end the only way to vary the stiffness profile in the longitudinal/axial direction of the catheter body (i.e., creating discrete region(s) of desired flexibility/stiffness) is by changing either picks per inch (PPI) of the reinforcing filament layer 120 and/or durometer of the material of the outer jacket 125. Transitions in stiffness achieved by variation in PPI of the reinforcing filament layer 120 and/or durometer of the material of the outer jacket 125 are abrupt (i.e., instantaneous, sudden or non-gradual) representing a planar transition, interface or boundary (i.e., a first stiffness on one side of the planar interface and a second stiffness on the other side). During advancement, the abrupt or sudden planar transition in stiffness realized by varying PPI and/or durometer of the material undesirably creates a kink point preventing successful and efficient navigation of the catheter through the vasculature anatomy.

The present disclosure recognizes several additional parameters that may be varied to realize a non-uniform stiffness profile in the longitudinal/axial direction producing a gradual transition (i.e., smoother) over an extended longitudinal/axial length, compared to the abrupt (i.e., instantaneous, sudden or non-gradual) planar interface or boundary. Specifically, a desired non-uniform stiffness profile in the longitudinal/axial direction may be realized by varying: (i) differential between maximum and minimum outer diameter of the body of the catheter; and/or (ii) ridge pitch. That is, the greater the differential between maximum and minimum outer diameter of the body the less stiff (i.e., more flexible) the body of the catheter; and the larger the ridge pitch 110 the less stiff (i.e., more flexible) the body of the catheter. Thus, without altering PPI of the reinforcing filament layer 120 or the durometer of the material of the outer jacket 125, a desired gradual (i.e., smooth) transition of non-uniform stiffness in the longitudinal/axial direction may be achieved by varying only the differential between maximum and minimum outer diameter of the catheter body and/or ridge pitch 113. In addition to these two new properties, a non-uniform stiffness (i.e., flexibility) may also be achieved by varying such conventional properties as PPI of the reinforcing filament layer 120 and/or durometer of the material of the outer jacket 125.

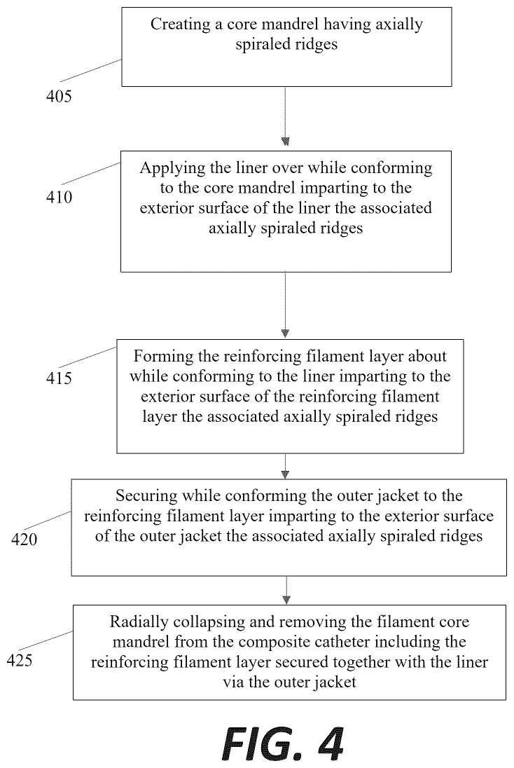

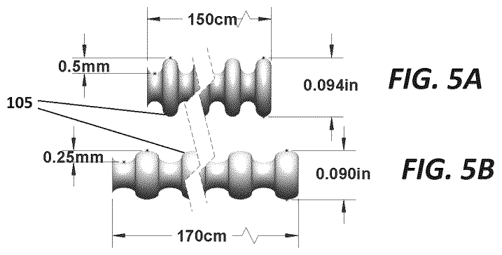

FIG. 4 is an exemplary flow chart of the method of manufacture of the body 100 of the catheter in accordance with the present disclosure having a plurality of axially spiraled ridges 110 creating an outer profile non-uniform in outer diameter in the longitudinal/axial direction. Initially, in step 405 the core mandrel 105 is created (otherwise a pre-formed version is provided) having a plurality of axially spiraled ridges 110. In step 410, the liner 115 is applied over while confirming to the core mandrel 105 taking on, adopting, identically matching, conforming, imparting or configured to the exterior surface of the liner the associated axially spiraled ridges 110. Next, in step 415, reinforcing filament layer 120 (e.g., metal wire(s) or polymer fiber(s), that are either braided/woven or coiled) is formed about while conforming to the liner 115 imparting to the exterior surface of the reinforcing filament layer 120 the associated axially spiraled ridges 110. The reinforcing filament layer 120 and liner 115 in step 420 are secured together (e.g., fused when subject to heat) via the outer jacket 125 imparting to the exterior surface of the outer jacket 125 the associated axially spiraled ridges 110 while preventing unraveling of the reinforcing filament layer 120. FIGS. 2A & 2B depict longitudinal/axial and radial cross-sectional views, respectively, of the various layers of the catheter body assembled about the core mandrel in accordance with the present disclosure. The last step 425 in the manufacture of the body 100 of the catheter is radially collapsing and removing (i.e., withdrawing) the core mandrel 105 from the composite catheter including the reinforcing filament layer 120 secured together with the liner 115 via the outer jacket 125. The composite catheter with the core mandrel 105 removed is shown in FIGS. 3A & 3B. Radial collapse of the core mandrel 105 may be accomplished by pulling in opposing directions (e.g., proximal and distal directions) on pull wires 200a, 200b secured at its respective proximal and distal ends of the core mandrel 105. The core mandrel 105 is illustrated in FIG. 5A in a radially non-collapsed state, i.e., before being pulled in opposing directions by pull wires 200a, 200b in opposite directions, while FIG. 5B depicts the core mandrel 105 in a radially collapsed state while being pulled in opposing directions by pull wires 200a, 200b allowing withdraw. By way of non-limiting illustrative example, the core mandrel 105 in the radially non-collapsed state has a maximum outer diameter of approximately 0.094 in., a differential in maximum and minimum outer diameter of approximately 0.5 mm, and a longitudinal/axial length of approximately 150 cm (FIG. 5A). This same illustrative example core mandrel in the radially collapsed state, in FIG. 5B, has a maximum outer diameter of 0.090 in., a differential in maximum and minimum outer diameter of approximately 0.25 mm, and a longitudinal/axial length of approximately 170 cm. Following removal or withdraw of the radially collapsed core mandrel 105, what remains is the manufactured composite body 100 of the catheter (i.e., the assembled layers including the reinforcing filament layer 120 and the liner 115 secured via the outer jacket 125). In accordance with the present disclosure, an exterior surface of each respective layer (e.g., liner 115, reinforcing filament layer 120 and the outer jacket 125) comprising the manufactured composite body 100 of the catheter maintains the axially spiraled ridges 110 producing an outer profile having a non-uniform outer diameter in the longitudinal/axial direction. Accordingly, the axially spiraled ridges 110 are preserved with the application of each additional layer and are present in the outer profile of the manufactured composite body 100 of the catheter. As previously mentioned, the presence of the axially spiraled ridges 110 produces an outer profile of the manufactured composite body 100 of the catheter having a non-uniform outer diameter in the longitudinal/axial direction. Thus, only at the axially spiraled ridges 110 is the outer surface of the manufactured composite body 100 of the catheter in contact with either the inner wall of the vessel or inner wall of a lumen of another catheter (e.g., guide catheter or delivery catheter), minimizing friction forces generated therebetween and ease of trackability.

Optionally, the body 100 of the catheter including axially spiraled ridges producing an outer profile that is non-uniform in outer diameter in the longitudinal/axial direction may be intentionally configured to have a non-uniform stiffness (i.e., multiple discrete axial regions of differing stiffness or flexibility) in the longitudinal/axial direction by varying: (i) the differential between the maximum outer diameter and the minimum outer diameter of the body; and/or (ii) ridge pitch. That is, the greater the differential between maximum outer diameter and minimum outer diameter of the body 100 of the catheter or greater the ridge pitch 113 the less stiff (i.e., more flexible) that region. One advantageous example has maximum flexibility (i.e., less stiff) in a distal section (including the distal end/tip) of the body of the catheter fostering distal advancement through the vasculature anatomy, while proximally thereto the remaining portion of the body of the catheter is stiffer (i.e., less flexible) to push on the proximal end. While yet another example provides maximum flexibility (i.e., minimum stiff) in a distal section (including the distal end/tip) of the body 100 of the catheter assisting in distal advancement through the vasculature anatomy and minimum flexibility (i.e., maximum stiffness) in an opposite proximal section (including the proximal end/tip), while interposed therebetween is at least one intermediate axial section having an associated stiffness greater than the minimum stiffness in the distal section but less than the maximum stiffness in the proximal section.

Aspects of the present disclosure are also provided by the following numbered Clauses:
Clause 1: A catheter comprising: a body (100) having a proximal end (100a), an opposite distal end (100b) and an outer wall (100c) extending axially therebetween, the outer wall (100c) defining axially spiraled ridges (110) forming an undulating outer profile non-uniform in outer diameter in an axial direction.
Clause 2: The catheter of Clause 1, wherein the body (100) comprises: a liner (115); a reinforcing filament layer (120) disposed about the liner (115); and an outer jacket (125) covering the reinforcing filament layer (120); wherein each of the liner (115), the reinforcing filament layer (120) and the outer jacket (125) is identically configured to have the axially spiraled ridges (110) forming the undulating outer profile non-uniform in outer diameter in the axial direction as defined in the outer wall (100c) of the body (100).
Clause 3: The catheter of Clause 2, wherein the reinforcing filament layer (120) is braided or coiled.
Clause 4: The catheter of any of Clauses 2 through 3, wherein the outer jacket (125) is dip coated or wrapped about the reinforcing filament layer (120).
Clause 5: The catheter of any of Clauses 2 through 4, wherein the liner (115) is polytetrafluoroethylene and the outer jacket (125) is a polymer.
Clause 6: The catheter of any of Clauses 1 through 5, wherein the undulating outer profile extends axially from substantially the proximal end (100a) to substantially the distal end (100b) of the body of the catheter (100).
Clause 7: The catheter of any of Clauses 1 through 6, wherein the body (100) has a non-uniform stiffness profile in the axial direction by varying: (i) differential between a maximum outer diameter and a minimum outer diameter of the body (100); and/or (iii) ridge pitch, wherein the ridge pitch is an axial distance between the axially spiraled ridges (110) adjacent to one another in the axial direction.
Clause 8: The catheter of Clause 7, wherein the greater the differential between the maximum outer diameter and the minimum outer diameter and/or the larger the ridge pitch, the less stiff the body (100) of the catheter.
Clause 9: A method for manufacturing a catheter having a body (100) including a liner (115), a reinforcing filament layer (120) and an outer jacket (125); the method comprising the steps of: creating a core mandrel (105) having a proximal end (105a), an opposite distal end (105b) and an outer wall (105c) extending axially therebetween; the core mandrel (105) defining axially spiraled ridges (110) along the outer wall forming an undulating outer profile non-uniform in outer diameter in an axial direction; and applying the liner (115) over the core mandrel (105) while conforming to without altering the axially spiraled ridges (110) and the undulating outer profile non-uniform in outer diameter in the axial direction; forming the reinforcing filament layer (120) about the liner (115) while conforming to without altering the axially spiraled ridges (110) and the undulating outer profile non-uniform in outer diameter in the axial direction; fusing the outer jacket (125) to the reinforcing filament layer (120) while conforming to without altering the axially spiraled ridges (110) and the undulating outer profile non-uniform in outer diameter in the axial direction; and radially collapsing and removing the core mandrel (105) from the body (100) including the liner (115), the reinforcing filament layer (120) and the outer jacket (125).
Clause 10: The method of Clause 9, further comprising establishing a non-uniform stiffness profile in the axial direction by varying: (i) differential between a maximum outer diameter and a minimum outer diameter of the body (100); and/or (iii) ridge pitch, wherein the ridge pitch is an axial distance between the axially spiraled ridges (110) adjacent to one another in the axial direction.
Clause 11: The method of Clause 10, wherein the greater the differential between the maximum outer diameter and the minimum outer diameter and/or the higher the ridge pitch, the less stiff the body (100) of the catheter.
Clause 12: The method of any of Clauses 9 through 11, wherein the reinforcing filament layer (120) is braided or coiled.
Clause 13: The method of any of Clauses 9 through 12, wherein the outer jacket (125) is dip coated or wrapped about the reinforcing filament layer (120).
Clause 14: The method of any of Clauses 9 through 13, wherein the liner (115) is polytetrafluoroethylene and the outer jacket (125) is a polymer.
Clause 15: The method of any of Clauses 9 through 14, wherein the undulating outer profile extends axially from substantially the proximal end (100a) to substantially the distal end (100b) of the body (100) of the catheter.
Clause 16: A method for using a catheter during endovascular treatment at a target site in a vessel; the catheter including a body (100) having a proximal end (100a), an opposite distal end (100b) and an outer wall (100c) extending axially therebetween, the outer wall (100c) defining axially spiraled ridges (110) forming an undulating outer profile non-uniform in outer diameter in an axial direction; the method comprising the step of: navigating the catheter to the target site; wherein the undulating outer profile non-uniform in outer diameter in the axial direction of the outer wall of the catheter is subject to contact surface friction forces only at the axially spiraled ridges.
Clause 17: The method of Clause 16, wherein the contact surface friction forces are produced by the axially spiraled ridges being in direct surface contact with an inner wall of the vessel.
Clause 18: The method of any of clauses 16 through 17, wherein prior to the navigating step, further comprising advancing a guide catheter to the target site in the vessel; and the navigating step comprising navigating the catheter through the guide catheter to the target site; wherein the contact surface friction forces are produced by the axially spiraled ridges being in direct surface contact with an inner wall of the guide catheter.
Clause 19: The method of any of Clauses 16 through 18, wherein the body (100) comprises: a liner (115); a reinforcing filament layer (120) disposed about the liner (115); and an outer jacket (125) covering the reinforcing filament layer (120); wherein each of the liner (115), the reinforcing filament layer (120) and the outer jacket (125) is identically configured to have the axially spiraled ridges (110) forming the undulating outer profile non-uniform in outer diameter in the axial direction as defined in the outer wall (100c) of the body (100).

Clause 20: The method of any of clauses 16 through 19, wherein the reinforcing filament layer (120) is braided or a coil.

The descriptions contained herein are examples of embodiments of the invention and are not intended in any way to limit the scope of the invention. As described herein, the invention contemplates many variations and modifications of a catheter, method of manufacturing a catheter, and method of using a catheter, wherein the body of the catheter includes axially spiraled ridges producing an outer profile having a non-uniform outer diameter in the longitudinal/axial direction. Trackability is fostered by minimizing friction forces resulting from contact of the exterior surface of the body of the catheter with the inner wall of the vessel or inner wall of another catheter (e.g., guide catheter or delivery catheter) exclusively or only at the axially spiraled ridges. In addition, a non-uniform stiffness profile in the longitudinal/axial direction of the body of the catheter may be realized by varying: (i) differential between maximum and minimum outer diameter of the body of the catheter; and/or (ii) ridge pitch representing distance in the longitudinal/axial direction between adjacent axially spiraled ridges. Modifications and variations apparent to those having skilled in the pertinent art according to the teachings of this disclosure are intended to be within the scope of the claims which follow.

What is claimed is:

1. A catheter comprising:
a body having a proximal end, an opposite distal end and an outer wall extending axially therebetween, the outer wall defining axially spiraled ridges forming an undulating outer profile non-uniform in outer diameter in an axial direction.

2. The catheter in accordance with claim 1, wherein the body comprises:
a liner;
a reinforcing filament layer disposed about the liner; and
an outer jacket covering the reinforcing filament layer;
wherein each of the liner, the reinforcing filament layer and the outer jacket is identically configured to have the axially spiraled ridges forming the undulating outer profile non-uniform in outer diameter in the axial direction as defined in the outer wall of the body.

3. The catheter in accordance with claim 2, wherein the reinforcing filament layer is braided or coiled.

4. The catheter in accordance with claim 2, wherein the outer jacket is dip coated or wrapped about the reinforcing filament layer.

5. The catheter in accordance with claim 2, wherein the liner is polytetrafluoroethylene and the outer jacket is a polymer.

6. The catheter in accordance with claim 1, wherein the undulating outer profile extends axially from substantially the proximal end to substantially the distal end of the body of the catheter.

7. The catheter in accordance with claim 1, wherein the body has a non-uniform stiffness profile in the axial direction by varying: (i) differential between a maximum outer diameter and a minimum outer diameter of the body; and/or (iii) ridge pitch, wherein the ridge pitch is an axial distance between the axially spiraled ridges adjacent to one another in the axial direction.

8. The catheter in accordance with claim 7, wherein the greater the differential between the maximum outer diameter and the minimum outer diameter and/or the larger the ridge pitch, the less stiff the body of the catheter.

9. A method for manufacturing a catheter having a body including a liner, a
reinforcing filament layer and an outer jacket; the method comprising the steps of:
creating a core mandrel having a proximal end, an opposite distal end and an outer wall extending axially therebetween; the core mandrel defining axially spiraled ridges along the outer wall forming an undulating outer profile non-uniform in outer diameter in an axial direction; and
applying the liner over the core mandrel while conforming to without altering the axially spiraled ridges and the undulating outer profile non-uniform in outer diameter in the axial direction;
forming the reinforcing filament layer about the liner while conforming to without altering the axially spiraled ridges and the undulating outer profile non-uniform in outer diameter in the axial direction;
fusing the outer jacket to the reinforcing filament layer while conforming to without altering the axially spiraled ridges and the undulating outer profile non-uniform in outer diameter in the axial direction; and
radially collapsing and removing the core mandrel from the body including the liner, the reinforcing filament layer and the outer jacket.

10. The method in accordance with claim 9, further comprising establishing a non-uniform stiffness profile in the axial direction by varying: (i) differential between a maximum outer diameter and a minimum outer diameter of the body; and/or (iii) ridge pitch, wherein the ridge pitch is an axial distance between the axially spiraled ridges adjacent to one another in the axial direction.

11. The method in accordance with claim 10, wherein the greater the differential between the maximum outer diameter and the minimum outer diameter and/or the higher the ridge pitch, the less stiff the body of the catheter.

12. The method in accordance with claim 9, wherein the reinforcing filament layer is braided or coiled.

13. The method in accordance with claim 9, wherein the outer jacket is dip coated or wrapped about the reinforcing filament layer.

14. The method in accordance with claim 9, wherein the liner is polytetrafluoroethylene and the outer jacket is a polymer.

15. The method in accordance with claim 9, wherein the undulating outer profile extends axially from substantially the proximal end to substantially the distal end of the body of the catheter.

16. A method for using a catheter during endovascular treatment at a target site in a vessel; the catheter including a body having a proximal end, an opposite distal end and an outer wall extending axially therebetween, the outer wall defining axially spiraled ridges forming an undulating outer profile non-uniform in outer diameter in an axial direction; the method comprising the step of:
navigating the catheter to the target site; wherein the undulating outer profile non-uniform in outer diameter in the axial direction of the outer wall of the catheter is subject to contact surface friction forces only at the axially spiraled ridges.

17. The method in accordance with claim 16, wherein the contact surface friction forces are produced by the axially spiraled ridges being in direct surface contact with an inner wall of the vessel.

18. The method in accordance with claim 16, wherein prior to the navigating step, further comprising advancing a guide catheter to the target site in the vessel; and the navigating step comprising navigating the catheter through the guide catheter to the target site; wherein the contact surface friction forces are produced by the axially spiraled ridges being in direct surface contact with an inner wall of the guide catheter.

19. The method in accordance with claim 16, wherein the body comprises:
a liner;
a reinforcing filament layer disposed about the liner; and
an outer jacket covering the reinforcing filament layer;
wherein each of the liner, the reinforcing filament layer and the outer jacket is identically configured to have the axially spiraled ridges forming the undulating outer profile non-uniform in outer diameter in the axial direction as defined in the outer wall of the body.

20. The method in accordance with claim 19, wherein the reinforcing filament layer is braided or a coil.
